(19) United States
(12) Patent Application Publication
(10) Pub. No.: US 2025/0134550 A1
(43) Pub. Date: May 1, 2025

(54) ATHERECTOMY SYSTEM WITH PERFORMANCE TRACKING

(71) Applicant: Boston Scientific Scimed, Inc., Maple Grove, MN (US)

(72) Inventors: Daniel Frank Massimini, Brooklyn Park, MN (US); Scott Hansen, Maple Grove, MN (US); Holden Tranquillo, Arden Hills, MN (US); Andrew David Bicek, Elk River, MN (US)

(73) Assignee: Boston Scientific Scimed, Inc., Maple Grove, MN (US)

(21) Appl. No.: 18/929,980

(22) Filed: Oct. 29, 2024

Related U.S. Application Data
(60) Provisional application No. 63/594,296, filed on Oct. 30, 2023.

(51) Int. Cl. A61B 17/3207; A61B 17/00

(52) U.S. Cl. CPC A61B 17/320758; A61B 2017/00057; A61B 2017/00075

(57) ABSTRACT

An atherectomy system includes a housing and a drive mechanism disposed within the housing, the drive mechanism including a knob that allows translation of the drive mechanism relative to the housing. A sensor is adapted to output a signal representative of a current position of the knob relative to the housing. A controller is adapted to receive the signal representative of the current position of the knob. The controller may be adapted to quantify operation of the atherectomy system.

### CROSS-REFERENCE TO RELATED APPLICATIONS

This application claims the benefit of priority under 35 U.S.C. § 119 of U.S. Provisional Application No. 63/594,296, filed Oct. 30, 2023, the entire disclosure of which is hereby incorporated by reference.

### TECHNICAL FIELD

The present disclosure pertains to medical devices, and methods for manufacturing and using medical devices. More particularly, the present disclosure pertains to rotational medical devices, methods, and systems.

### BACKGROUND

A wide variety of intracorporeal medical devices have been developed for medical use, for example, intravascular use. Some of these devices include guidewires, catheters, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### SUMMARY

This disclosure provides design, material, manufacturing method, and use alternatives for medical devices. An example may be found in an atherectomy system. The atherectomy system includes a housing, a drive assembly adapted to translate relative to the housing, a knob extending from the drive assembly such that translating the knob results in the drive assembly translating relative to the housing, and a sensor adapted to detect a current position of the knob relative to the housing.

Alternatively or additionally, the atherectomy system may further include a driveshaft operably coupled with the drive assembly, the driveshaft translating relative to the housing as the drive assembly translates relative to the housing, and an atherectomy burr operably coupled with the driveshaft.

Alternatively or additionally, the knob may provide feedback to the user as the user moves the knob back and forth relative to the housing, and the sensor may be further adapted to detect the current position of the knob relative to the housing without causing a noticeable change to the feedback provided by the knob.

Alternatively or additionally, the sensor may include a magnetic sensor.

Alternatively or additionally, the sensor may include a magnet disposed proximate the knob such that the magnet moves with the knob and a variable resistance strip adapted to output a variable voltage in response to movement of the magnet.

Alternatively or additionally, the magnet may be disposed within the knob.

Alternatively or additionally, the magnet disposed proximate the knob may include a first magnet, and the variable resistance strip may include an entrapped second magnet that moves within the variable resistance strip in accordance with movement of the first magnet.

Alternatively or additionally, the variable resistance strip may output a variable voltage as a result of movement of the entrapped second magnet.

Alternatively or additionally, the sensor may include a light-based sensor.

Alternatively or additionally, the sensor may include a visual-based sensor.

Another example may be found in an atherectomy system. The atherectomy system includes a housing, a drive assembly disposed within the housing and including a knob that allows translation of the drive assembly relative to the housing, a sensor adapted to output a signal representative of a current position of the control knob relative to the housing, and a controller adapted to receive the signal representative of the current position of the knob.

Alternatively or additionally, the controller may be further adapted to track the current position of the knob over time.

Alternatively or additionally, the controller may be further adapted to track a rotational speed of the drive assembly over time.

Alternatively or additionally, the controller may be further adapted to output data indicative of how a user is operating the atherectomy system over time.

Alternatively or additionally, the sensor may include a magnetic sensor.

Alternatively or additionally, the sensor may include a first magnet disposed proximate the knob so that the first magnet moves as the control knob moves, a variable resistance strip disposed proximate the housing, and a second magnet trapped within the variable resistance strip and movable relative to the variable resistance strip in response to movement of the first magnet. Movement of the second magnet relative to the variable resistance strip causes the variable resistance strip to output a variable voltage representative of a current position of the knob.

Another example may be found in an atherectomy system. The atherectomy system includes a housing, a drive assembly adapted to translate relative to the housing, a knob extending from the drive assembly such that translating the knob results in the drive assembly translating relative to the housing, the knob providing feedback to the user as the user translates the knob, and a sensor adapted to detect movement of the knob without causing a noticeable change to the feedback provided by the knob.

Alternatively or additionally, the atherectomy system may further include a driveshaft operably coupled with the drive assembly, the driveshaft translating relative to the housing as the drive assembly translates relative to the housing, and an atherectomy burr operably coupled with the driveshaft.

Alternatively or additionally, the atherectomy system may further include a controller that is adapted to receive a signal from the sensor representative of movement of the knob and use that signal to quantify operation of the atherectomy system.

Alternatively or additionally, the controller may be further adapted to output data indicative of how a user is operating the atherectomy system over time.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The invention may be more completely understood in consideration of the following detailed description of various embodiments of the invention in connection with the accompanying drawings, in which:

FIG. 1 is a schematic diagram showing an illustrative atherectomy system;

FIG. 2 is a schematic diagram showing features of the illustrative atherectomy system of FIG. 1;

FIG. 3 is a schematic block diagram showing features of the illustrative atherectomy system of FIG. 1;

FIGS. 4A, 4B and 4C are schematic block diagrams showing illustrative sensors usable in the illustrative atherectomy system of FIG. 1; and

FIG. 5 is a schematic block diagram showing an example of an illustrative sensor usable in the illustrative atherectomy system of FIG. 1.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The drawings, which are not necessarily to scale, depict illustrative embodiments and are not intended to limit the scope of the invention.

Cardiovascular disease and peripheral arterial disease may arise from accumulation of atheromatous material on the inner walls of vascular lumens, resulting in a condition known as atherosclerosis. Atheromatous and other vascular deposits may restrict blood flow and can cause ischemia in a heart of a patient, vasculature of a patient's legs, a patient's carotid artery, etc. Such ischemia may lead to pain, swelling, wounds that will not heal, amputation, stroke, myocardial infarction, and/or other conditions.

Atheromatous deposits may have widely varying properties, with some deposits being relatively soft and others being fibrous and/or calcified. In the latter case, the deposits may be referred to as plaque. Atherosclerosis occurs naturally as a result of aging, but may also be aggravated by factors such as diet, hypertension, heredity, vascular injury, and the like. Atherosclerosis may be treated in a variety of ways, including drugs, bypass surgery, and/or a variety of catheter-based approaches that may rely on intravascular widening or removal of the atheromatous or other material occluding the blood vessel. Atherectomy is a catheter-based intervention that may be used to treat atherosclerosis.

Atherectomy is an interventional medical procedure performed to restore a flow of blood through a portion of a patient's vasculature that has been blocked by plaque or other material (e.g., blocked by an occlusion). In an atherectomy procedure, a device on an end of a drive shaft that is used to engage and/or remove (e.g., abrade, grind, cut, shave, etc.) plaque or other material from a patient's vessel (e.g., artery or vein). In some cases, the device on an end of the drive shaft may be abrasive and/or may otherwise be configured to remove plaque from a vessel wall or other obstruction in a vessel when the device is rotating and engages the plaque or other obstruction. In some cases, atherectomy involves using an abrasive atherectomy burr that is rotated at high speeds exceeding 100,000 revolutions per minute (RPM) in order to abrade plaque and other hardened materials from within the patient's vessel. Atherectomy burrs may be rotated at speeds exceeding 140,000 RPM, at speeds exceeding 180,000 RPM and even at speeds as high as 220,000 RPM. Atherectomy may include orbital atherectomy in addition to rotational atherectomy.

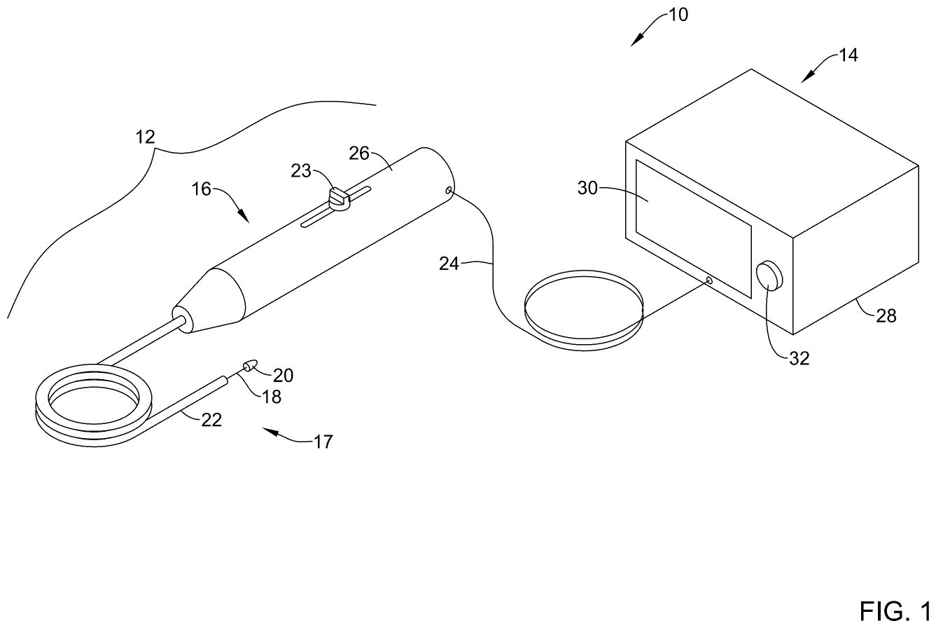

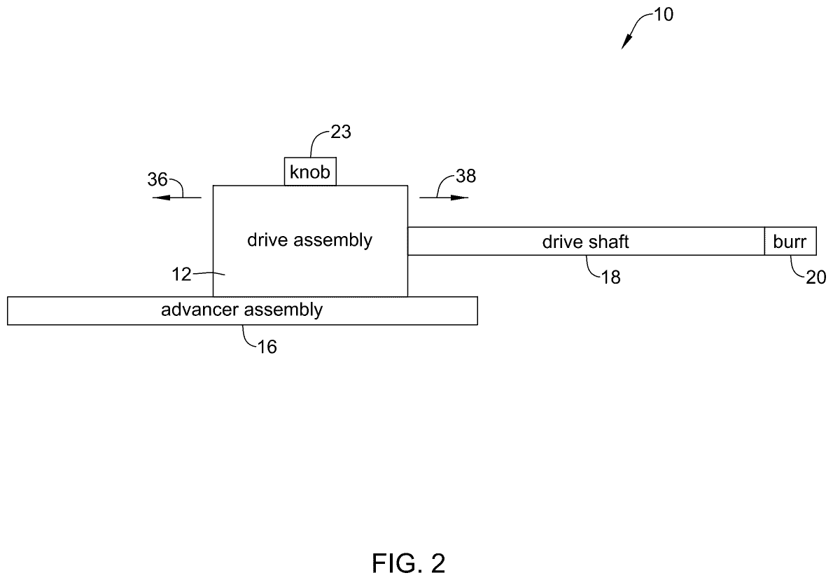

FIGS. 1 and 2 depict an atherectomy system 10. The atherectomy system 10 may be electrically driven, pneumatically driven and/or driven in one or more other suitable manners. Additional or alternative components to those illustrated and described herein may be utilized in the operation of the atherectomy system 10. The atherectomy system 10 may include a drive assembly 12 and a control unit 14 (e.g., a controller). The drive assembly 12 may include, among other elements, an advancer assembly 16 and a rotation assembly 17. Although the control unit 14 is depicted as being separate from the drive assembly 12 in FIG. 1, the functionality of the control unit 14 and the drive assembly 12 may be incorporated into a single component (e.g., in the advancer assembly 16 or other suitable single component).

The rotation assembly 17 may include a drive shaft 18 (e.g., an elongate member that may be or may include a flexible driveshaft or other suitable driveshaft), an atherectomy burr 20 and an elongate member 22 having a first end (e.g., a proximal end), a second end (e.g., a distal end), and a lumen extending from the first end to the second end for receiving the drive shaft 18. In some cases, the elongate member 22 may be an elongated tubular member. The atherectomy burr 20 may have a rough or sharp surface, such that it is configured to grind, abrade, cut, shave, etc. plaque from a vessel wall or other obstruction in a vessel when it is rotated.

The advancer assembly 16 may include a knob 23, a housing 26, the drive assembly 12 and/or one or more other suitable components. In some instances, the drive assembly 12 may be or may include a motor (e.g., an electric motor, pneumatic motor, or other suitable motor) at least partially housed within the housing 26 and in communication with the knob 23, the drive shaft 18, and the control unit 14. In some cases, the motive force may not be disposed within the drive assembly 12, but may instead be remotely located, with a flexible drive cable extending from the motive force to the drive assembly 12. The knob 23 may be configured to advance along a longitudinal path to longitudinally advance the drive assembly 12 and the rotation assembly 17. The housing 26 may at least partially house the drive assembly 12 and the knob 23 may be at least partially accessible from an exterior of the housing 26.

In some instances, the drive assembly 12 is adapted to be translationally secured relative to an advancer assembly 16. In some instances, the advancer assembly 14 may be adapted to be fixed in space, such as being secured to a table, for example. In some instances, the advancer assembly 16 may be part of an advancer housing such as the housing 26. The drive assembly 12 may also be disposed within an advancer handle, for example, but is adapted to translate back and forth (left and right in the illustrated orientation) as indicated by arrows 36 and 38 in response to a user moving the knob 23 in the directions indicated by the arrows 36 and 38. In some instances, as the drive assembly 12 moves back and forth, the drive shaft 18 also moves correspondingly.

The drive assembly 12 may be coupled to the drive shaft 18 in a suitable manner including, but not limited to, a weld connection, a clamping connection, an adhesive connection, a threaded connection, and/or other suitable connection configured to withstand rotational speeds and forces. The drive shaft 18 may be formed from one or more of a variety of materials. For example, the drive shaft 18 may be formed from one or more of a variety of materials, including steel, stainless steel, other metal, polymer, and/or other suitable materials. The drive shaft 18 may have a suitable diameter and/or length for traversing vasculature of a patient. The diameter and/or the length of the drive shaft 18 may depend on the dimension of the lumen of the elongate member 22, the dimensions of vessels of a patient to be traversed, and/or one or more other suitable factors. In some cases, the drive shaft 18 may have a diameter in a range from about 0.030 centimeters (cm) or smaller to about 0.150 cm or larger and a working length in a range from about ten (10) cm or shorter to about three hundred (300) cm or longer. In one example, the drive shaft 18 may have a diameter of about 0.07493 cm and a length of about one hundred forty seven (147) cm. Alternatively, the drive shaft 18 may have a different suitable diameter and/or different suitable length.

The atherectomy burr 20 may have an outer perimeter which is equal to or larger than a distal diameter of the drive shaft 18 and/or the elongate member 22. Alternatively or in addition, the atherectomy burr 20 may have an outer perimeter which is smaller than a diameter of the drive shaft 18 and/or the elongate member 22. The atherectomy burr 20 may be coupled to the drive shaft 18. Where the drive shaft 18 has a first end portion (e.g., a proximal end portion) and a second end portion (e.g., a distal end portion), the atherectomy burr 20 may be coupled to the drive shaft 18 at or near the second end portion. In some cases, the atherectomy burr 20 may be located at or adjacent a terminal end of the second end portion of the drive shaft 18. The atherectomy burr 20 may be coupled to the drive shaft 18 in any manner. For example, the atherectomy burr 20 may be coupled to the drive shaft 18 with an adhesive connection, a threaded connection, a weld connection, a clamping connection, and/or other suitable connection configured to withstand rotational speeds and forces. Similar to as discussed above with respect to the connection between the drive shaft 18 and the drive mechanism, as the drive shaft 18 and/or the atherectomy burr 20 may rotate at speeds between zero (0) RPM and 250,000 RPM or higher, the coupling between the drive shaft 18 and the atherectomy burr 20 may be configured to withstand such rotational speeds and associated forces.

The drive assembly 12 and the control unit 14 may be in communication and may be located in or may have a same housing and/or located in or have separate housings (e.g., the advancer assembly housing 26 and a control unit housing 28 or other housings). Whether in the same housing or in separate housings, the drive assembly 12 and the control unit 14 may be in communication through a wired connection (e.g., via one or more wires in an electrical connector 24 or other suitable electrical connector) and/or a wireless connection. Wireless connections may be made via one or more communication protocols including, but not limited to, cellular communication, ZigBee, Bluetooth, Wi-Fi, Infrared Data Association (IrDA), dedicated short range communication (DSRC), EnOcean, and/or any other suitable common or proprietary wireless protocol, as desired.

Although not necessarily shown in FIG. 1, the drive assembly 12 may include and/or enclose one or more operational features. For example, among other features, the drive assembly 12 may include a motor (e.g., as discussed above and/or other suitable motor), rubber feet, control electronics, drive circuitry, etc.

The control unit 14, which may be separate from the drive assembly 12 (e.g., as shown in FIG. 1) or may be included in the drive assembly 12, may include several features. For example, as shown in FIG. 1, the control unit 14 may include a display 30 and a control knob 32 (e.g., a motor speed (e.g., RPM or other speed) adjustment knob or other control knob). Additionally or alternatively, the control unit 14 may include one or more other features for controlling the drive mechanism and/or other features of the drive assembly 12 (e.g., one or more drive mechanism states of the drive mechanism) including, but not limited to, a processor, memory, input/output devices, a speaker, volume control buttons, on/off power supply switch, motor activation switch, a timer, a clock, and/or one or more other features.

In some cases, the control unit 14 may include one or more drive mechanism load output control mechanisms for controlling an operation of the atherectomy system 10. In one example of a drive mechanism load output control mechanism that may be included in the control unit 14, the control unit 14 may include a mechanism configured to set and/or adjust an advancing load output (e.g., a rotational speed) and/or a retracting load output from the drive assembly 12. Additionally or alternatively, the control unit 14 may include other control and/or safety mechanism for controlling the operation of the atherectomy system 10 and mitigating risks to patients.

In some instances, there may be benefits in being able to track how a user is operating the atherectomy system 10. In some instances, there may be benefits in being able to track how the user operates the knob 23, as translating the knob 23 back and forth moves the drive assembly 12, which in turn moves the drive shaft 18 and the atherectomy burr 20 either distally or proximally, depending on which direction the user moves the knob 23. In addition to tracking just the relative position of the knob 23, there may be benefits in tracking the relative position of the knob 23 over time, which can provide an indication of how aggressively the user is moving the atherectomy burr 20 into a lesion, for example. It will be appreciated that knowing position with respect to time means that it is simple to determine the velocity at which the knob 23 is being moved simply by taking a derivative of the position with respect to time. Taking a second derivative of the position with respect to time easily yields acceleration. One user may advance the atherectomy burr 20 slowly and gently. Another user may advance the atherectomy burr 20 more quickly, and/or with greater force.

In some instances, being able to review data showing how a particular user utilized the atherectomy system 10 for a particular patient, in combination for example with their notes on the procedure may be useful in ascertaining why a particular problem occurred during the procedure. In some instances, the data from a particular procedure may also include recorded rotation speeds of the atherectomy burr 20. As an example, a user may accidently pop the atherectomy burr 20 completely through a lesion, such that the atherectomy burr 20 is positioned distal of the lesion. Being able to review the data from that procedure may reveal that the user was particularly aggressive in how quickly and with how much force they advanced the atherectomy burr 20. As another example, a user may report that they spent an inordinate amount of time clearing a particular lesion. A review of the data from that procedure may reveal that the user was not aggressive enough, and advanced the atherectomy burr 20 too slowly, and/or with not enough force, such that the atherectomy burr 20 was simply spinning at the proximal side of the lesion. In some instances, data showing a successful procedure may be used in helping to train a person how to perform an atherectomy procedure, such as by comparing the data from the successful procedure with the actual live data obtained during the training procedure. In some instances, data from an unsuccessful procedure may be used in helping to train a person how to perform an atherectomy procedure by demonstrating what not to do.

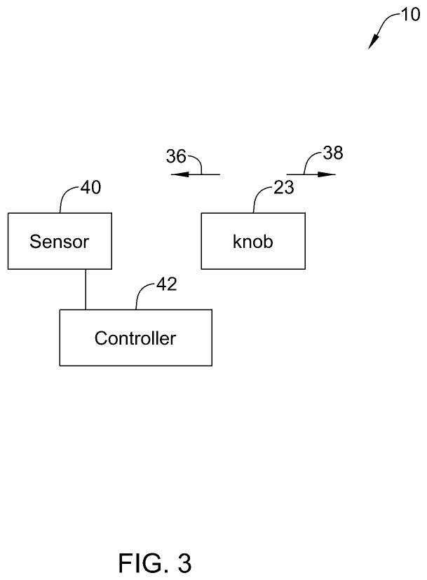

FIG. 3 is a schematic block diagram showing features of the illustrative atherectomy system 10. In some instances, the illustrative atherectomy system 10 may include a sensor 40 that is adapted to detect a current position of the knob 23 relative to the housing 26. In some instances, the knob 23 is adapted to provide feedback to the user as the user moves the knob 23 back and forth. The knob 23 moving easily can indicate that the atherectomy burr 20 is freely translating. The knob 23 moving with more difficulty, requiring additional force in order to cause the knob 23 to translate, can indicate that the atherectomy burr 20 has engaged a lesion. This feedback is important for the user to gain a better sense of what is happening at the atherectomy burr 20. In some instances, the sensor 40 may be adapted to detect the current position of the knob 23 relative to the housing 26 without causing a noticeable change to the feedback provided by the knob 23, and thus not impacting how the user uses the atherectomy system 10. In some instances, operation of the sensor 40 may be invisible to the user, regardless of whether the sensor 40 is visible to the user.

The atherectomy system 10 includes a controller 42. In some instances, the controller 42 may be located within the housing 26. In some instances, the controller 42 may be part of the control unit 14. In some instances, the controller 42, or at least the functionality of the controller 42, may be found within a controller forming part of the control unit 14. The controller 42 may be adapted to receive the signal representative of the current position of the knob 23 from the sensor 40. In some instances, the controller 42 may be further adapted to track the current position of the knob 23 over time. The controller 42 may be adapted to track a rotational speed of the drive assembly 12 over time, for example. In some instances, the controller 42 may be adapted to output data indicative of how a user is operating the atherectomy system 10 over time. The outputted data may be used for training new users. In some instances, the outputted data may be used for determining why a particular procedure was difficult, or had a poor outcome. In some instances, the controller 42 may track and record knob position over time as well as tracking and recording rotational speed of the drive assembly 12 over time.

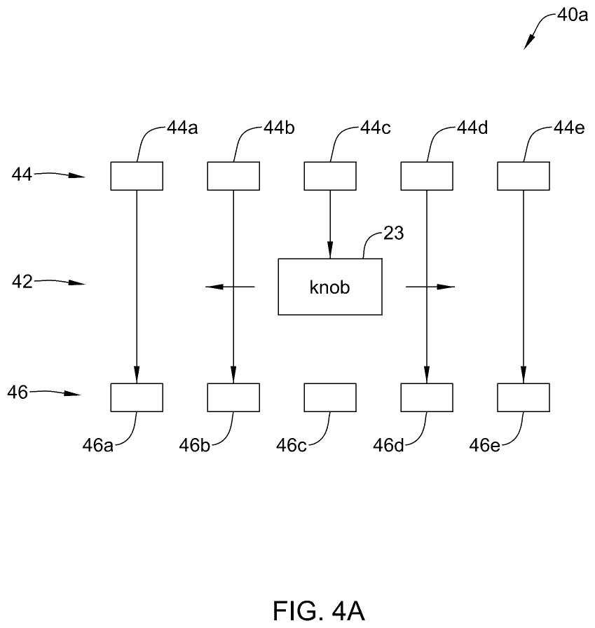

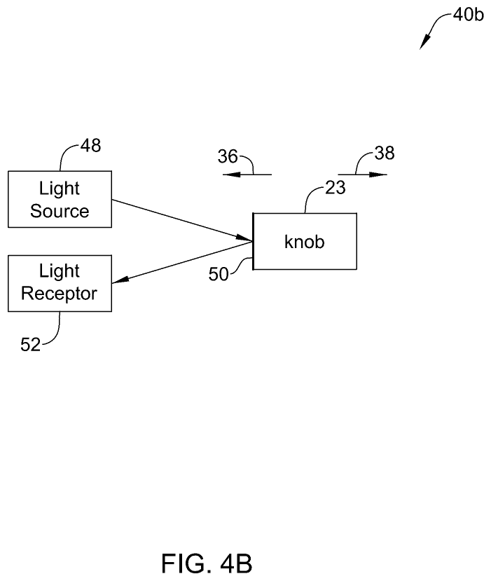

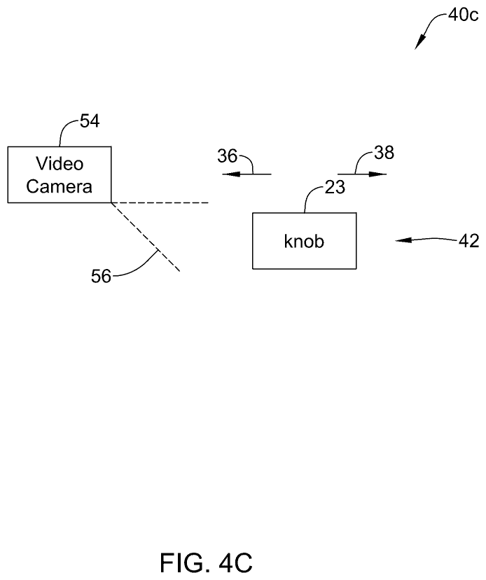

In some instances, the sensor 40 may be a light-based sensor. FIG. 4A is a schematic block diagram showing an illustrative sensor 40a that utilizes light to detect the position (and thus movement) of the knob 23. The sensor 40a allows the knob 23 to move along a path 42. The sensor 40a includes a number of light sources 44, individually labeled as 44a, 44b, 44c, 44d and 44c. While a total of five light sources 44 are shown, this is merely illustrative, as the sensor 40a may include any number of light sources 44. The light sources 44 may be LED lights, for example. The sensor 40a includes a corresponding number of light receptors 46, individually labeled as 46a, 46b, 46c, 46d and 46c. Each light source 44 will emit a light beam that is received by the corresponding light receptor 46, and may be blocked as the knob 23 moves along the path 42. As the individual light beams are blocked by the knob 23 as the knob 23 moves along that path, the sensor 40a may detect the current position of the knob 23 by virtue of which light beam(s) are blocked.

FIG. 4B shows another light-based sensor. FIG. 4B is a schematic block diagram showing an illustrative sensor 40b that utilizes light to detect the position (and thus movement) of the knob 23. A light source 48 may provide a light beam that strikes the knob 23. The light source 48 may include a laser, for example. In some instances, the knob 23 may include a reflective surface 50 that reflects the incident light beam. The reflected light beam may be detected by a light receptor 52. As the knob 23 moves back and forth along the path 42, the distance between the light source 48 and the reflective surface 50 will vary, resulting in differences in how quickly the light beam is reflected back. The changing length of time that it takes for a light beam to travel from the light source 48, reflect off the reflective surface 50, and return to the light receptor 52 provides an indication of the current position of the knob 23 along the path 42.

FIG. 4C is a schematic block diagram showing an illustrative sensor 40c that utilizes optics. The sensor 40c includes a video camera 54. The video camera 54 has a field of view 56 that includes the path 42 that the knob 23 travels along. In some instances, the video camera 54 continuously captures a video stream that includes the knob 23. In some instances, the video camera 54 may instead periodically capture a short video stream. In some instances, the video camera 54 may instead periodically capture still images of the knob 23. By using various video processing algorithms, the video camera 54 is able to ascertain the current position of the knob 23.

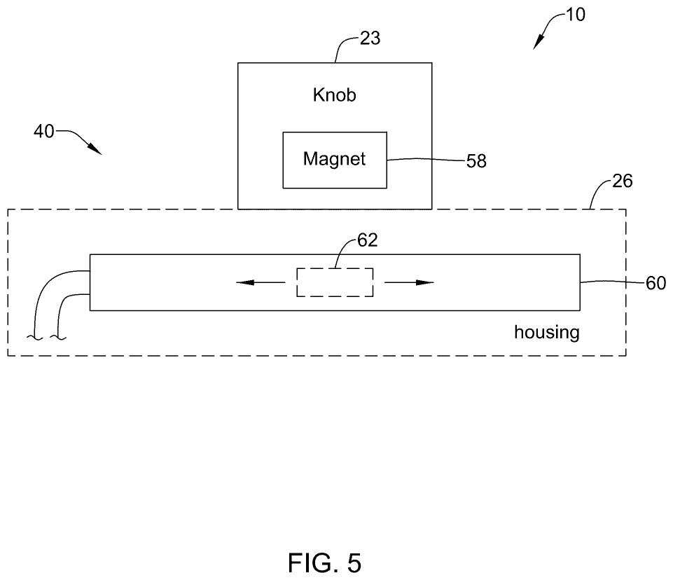

FIG. 5 is a schematic block diagram showing an example of the atherectomy system 10 in which the sensor 40 is a magnetic sensor. In some instances, a magnetic sensor may include a Hall effect sensor, an eddy current sensor or a fluxgate sensor. As additional examples, the sensor 40 may include a variable reluctance sensor or a linear variable differential transformer. As shown in FIG. 5, the sensor 40 may be considered as being a magnetic potentiometer, for example. In some instances, the sensor 40 includes a first magnet 58 that is disposed proximate the knob 23 such that the first magnet 58 moves as the knob 23 moves. The first magnet 58 may be a rare earth magnet, for example. In some instances, the first magnet 58 may be disposed inside of the knob 23. The first magnet 58 may be secured to an outer surface of the knob 23, for example. In some instances, the first magnet 58 may be secured relative to the advancer assembly 16, for example. The sensor 40 includes a variable resistance strip 60 that is adapted to output a variable voltage. In some instances, the variable resistance strip 60 includes an entrapped second magnet 62 that moves within the variable resistance strip 60 in response to movement of the first magnet 58. When the knob 23 (and hence the first magnet 58) moves to the left, the entrapped second magnet 62 moves to the left within the variable resistance strip 60. When the knob 23 (and hence the first magnet 58) moves to the right, the entrapped second magnet 62 moves to the right within the variable resistance strip 60. The variable resistance strip 60 outputs a variable voltage in response to movement of the entrapped second magnet 62. In some instances, the variable resistance strip 60 may be disposed along an inner surface of the housing 26, and thus is not visible. In some instances, the variable resistance strip 60 may be disposed along an outer surface of the housing 26. The variable resistance strip 60 may also be secured relative to other components within the housing 26, as long as those components are stationary with respect to the housing 26.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. An atherectomy system, comprising:
a housing;
a drive assembly adapted to translate relative to the housing;
a knob extending from the drive assembly such that translating the knob results in the drive assembly translating relative to the housing; and
a sensor adapted to detect a current position of the knob relative to the housing.

2. The atherectomy system of claim 1, further comprising:
a driveshaft operably coupled with the drive assembly, the driveshaft translating relative to the housing as the drive assembly translates relative to the housing; and
an atherectomy burr operably coupled with the driveshaft.

3. The atherectomy system of claim 1, wherein the knob provides feedback to the user as the user moves the knob back and forth relative to the housing, and the sensor is further adapted to detect the current position of the knob relative to the housing without causing a noticeable change to the feedback provided by the knob.

4. The atherectomy system of claim 1, wherein the sensor comprises a magnetic sensor.

5. The atherectomy system of claim 4, wherein the sensor comprises:
a magnet disposed proximate the knob such that the magnet moves with the knob; and
a variable resistance strip adapted to output a variable voltage in response to movement of the magnet.

6. The atherectomy system of claim 5, wherein the magnet is disposed within the knob.

7. The atherectomy system of claim 5, wherein the magnet disposed proximate the knob comprises a first magnet, and the variable resistance strip includes an entrapped second magnet that moves within the variable resistance strip in accordance with movement of the first magnet.

8. The atherectomy system of claim 7, wherein the variable resistance strip outputs a variable voltage as a result of movement of the entrapped second magnet.

9. The atherectomy system of claim 1, wherein the sensor comprises a light-based sensor.

10. The atherectomy system of claim 1, wherein the sensor comprises a visual-based sensor.

11. An atherectomy system, comprising:
a housing;
a drive assembly disposed within the housing and including a knob that allows translation of the drive assembly relative to the housing;
a sensor adapted to output a signal representative of a current position of the knob relative to the housing; and
a controller adapted to receive the signal representative of the current position of the knob.

12. The atherectomy system of claim 11, wherein the controller is further adapted to track the current position of the knob over time.

13. The atherectomy system of claim 12, wherein the controller is further adapted to track a rotational speed of the drive assembly over time.

14. The atherectomy system of claim 12, wherein the controller is further adapted to output data indicative of how a user is operating the atherectomy system over time.

15. The atherectomy system of claim 11, wherein the sensor comprises a magnetic sensor.

16. The atherectomy system of claim 11, wherein the sensor comprises:
a first magnet disposed proximate the knob so that the first magnet moves as the control knob moves;
a variable resistance strip disposed proximate the housing; and
a second magnet trapped within the variable resistance strip and movable relative to the variable resistance strip in response to movement of the first magnet;
wherein movement of the second magnet relative to the variable resistance strip causes the variable resistance strip to output a variable voltage representative of a current position of the knob.

17. An atherectomy system, comprising:
a housing;
a drive assembly adapted to translate relative to the housing;
a knob extending from the drive assembly such that translating the knob results in the drive assembly translating relative to the housing, the knob providing feedback to the user as the user translates the knob; and
a sensor adapted to detect movement of the knob without causing a noticeable change to the feedback provided by the knob.

18. The atherectomy system of claim 17, further comprising:
a driveshaft operably coupled with the drive assembly, the driveshaft translating relative to the housing as the drive assembly translates relative to the housing; and
an atherectomy burr operably coupled with the driveshaft.

19. The atherectomy system of claim 17, further comprising a controller that is adapted to receive a signal from the sensor representative of movement of the knob and use that signal to quantify operation of the atherectomy system.

20. The atherectomy system of claim 19. wherein the controller is further adapted to output data indicative of how a user is operating the atherectomy system over time.
